(19) United States
(12) Patent Application Publication
(10) Pub. No.: US 2016/0327622 A1
(43) Pub. Date: Nov. 10, 2016

(54) JOINT RECONSTRUCTION OF ACTIVITY AND ATTENUATION IN EMISSION TOMOGRAPHY USING MAGNETIC-RESONANCE-BASED PRIORS

(71) Applicant: General Electric Company, Schenectady, NY (US)

(72) Inventors: Sangtae Ahn, Guilderland, NY (US); Ravindra Mohan Manjeshwar, Glenville, NY (US); Florian Wiesinger, Freising (DE); Lishui Cheng, Schenectady, NY (US)

(21) Appl. No.: 14/847,643

(22) Filed: Sep. 8, 2015

Related U.S. Application Data
(60) Provisional application No. 62/157,188, filed on May 5, 2015.

(51) Int. Cl. G01R 33/48; A61B 5/00; A61B 5/055; G01T 1/164; G01T 1/29

(52) U.S. Cl. CPC G01R 33/481; G01T 1/1642; G01T 1/2985; G01T 1/2992; A61B 5/055; A61B 5/0035

(57) ABSTRACT

According to some embodiments, emission projection data and second source scan data are received. A prior map and a prior weight map are generated from second source scan data. A penalty function calculates voxel-wise differences between the prior map and a given image, transforms the voxel-wise differences and calculates a weighted sum of the transformed differences, using weights based on the prior weight map. Joint reconstruction of an emission image and an attenuation map proceeds iteratively and uses the penalty function.

### CROSS REFERENCE TO RELATED APPLICATION

This application claims the benefit of U.S. Provisional Patent Application No. 62/157,188 filed on May 5, 2015, the contents of which are hereby incorporated by reference for all purposes.

### BACKGROUND

The invention relates generally to tomographic imaging for medical applications and, more particularly, to methods and systems for joint reconstruction of activity and attenuation in emission tomography.

Attenuation correction is critical to accurate quantitation in positron emission tomography (PET). It has been proposed to use magnetic resonance (MR) imaging to aid in attenuation correction of PET images. The present inventors have recognized an opportunity for an improved manner of using an MR prior in joint reconstruction of activity and attenuation in a PET image.

### BRIEF DESCRIPTION

According to some embodiments, emission projection data and second source scan data corresponding to a subject are received. The second source scan data is from a mode of imaging different from emission projection imaging. Second source images are reconstructed based on the second source scan data. A prior map is generated based on the second source images. A prior weight map is generated based on the second source images. A penalty function is constructed. The penalty function calculates voxel-wise differences between the prior map and a given image. The penalty function also transforms each voxel-wise difference using a potential function. The penalty function further calculates a weighted sum of the transformed voxel-wise differences with weights for the weighted sum based on the prior weight map. An emission image and an attenuation map are reconstructed. The reconstruction of the emission image and the attenuation map includes iteratively updating the emission image based on the attenuation map and the emission projection data. The reconstruction of the emission image and the attenuation map also includes iteratively updating the attenuation map based on the emission image and the emission projection data by using the penalty function. A final attenuation map is obtained. A final emission image is generated.

Other embodiments are associated with systems and/or computer-readable medium storing instructions to perform any of the methods described herein.

### DRAWINGS

FIG. 1 is a pictorial view of a medical imaging system according to some embodiments.

FIG. 2 is a block diagram that represents aspects of the imaging system of FIG. 1.

FIGS. 3-5 are flow charts that illustrate a process that may be performed in the imaging system of FIG. 1.

FIGS. 6A-6C, 7A-7C, 8A-8C, 9A-9C, 10A-10C and 11A-11C are subject images that illustrate exemplary results of the process of FIGS. 3-5.

FIG. 12 is a block diagram of a computing system according to some embodiments.

### DETAILED DESCRIPTION

Embodiments disclosed herein include using an MR-based prior image in a synergistic manner in connection with joint reconstruction of activity and attenuation based on PET data. An attenuation map is generated based on MR image segmentation. The MR-based attenuation map is used as a MR-based prior and also as an initialization in joint reconstruction. The MR-based prior weight is spatially modulated to control the balance between MR segmentation-based attenuation and joint reconstruction. A small prior weight is used in low MR signal regions, which may include challenging areas such as implants, bones, internal air and lungs. For these areas there is a greater reliance on joint reconstruction. For other areas, a large prior weight may be used where MR can reliably recover fat and water. In addition, the prior weights may be spatially modulated depending on locations for robustness.

In some embodiments, the inclusion of the MR prior into the joint reconstruction involves use of a penalty function that utilizes an MR-based prior weight parameter.

The image processing approach disclosed herein may provide more flexibility and robustness that previously proposed PET/MR image processing techniques.

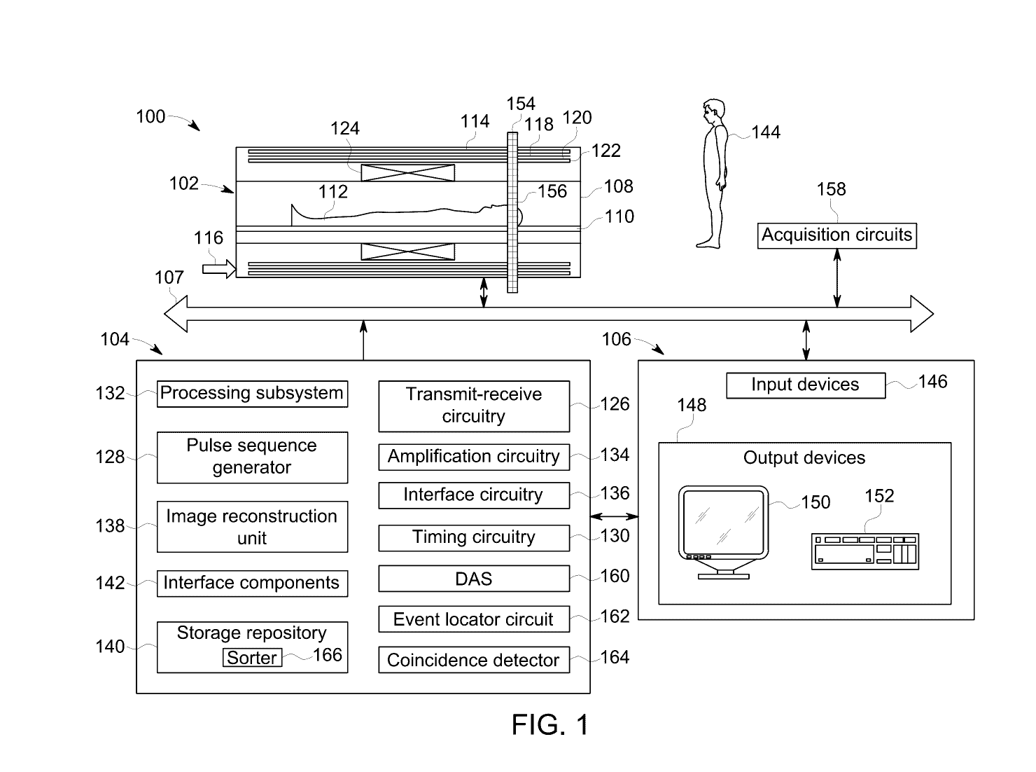

FIG. 1 illustrates an example imaging system 100 for enhanced imaging of a subject of interest according to some embodiments. The system 100 may correspond to a hybrid PET/MR system configured to generate an emission activity map and an attenuation map. Although the embodiment illustrated in FIG. 1 illustrates an integrated PET/MR system, in other embodiments independent PET and MR systems may be employed for imaging the subject.

The hybrid PET/MR imaging system 100 may include a scanner 102, a system controller 104 and an operator interface 106. The components 102, 104, 106 may be communicatively coupled to each other over a communications link and/or communication network 107. In some embodiments, the PET/MR system 100 may be configured to generate at least the MR images within the same repetition time (TR).

The embodiment depicted in FIG. 1 shows a full body scanner 102, but in other embodiments, one or both of the PET and MR scanning devices may employ other configurations, such as those suitable for imaging only one or more specific parts of the subject. In some embodiments, the scanner 102 may be configured to allow access by a physician, such as during interventional imaging.

In some embodiments, the scanner 102 may include a patient bore 108 into which a table 110 may be positioned for disposing the subject such as a patient 112 in a desired position for scanning. The scanner 102 may include a series of associated coils for imaging the patient 112. In some embodiments, the scanner 102 includes a primary magnet coil 114, for example, energized via a power supply 116 for generating a primary magnetic field generally aligned with the patient bore 108. The scanner 102 may further include a series of gradient coils 118, 120 and 122 grouped in a coil assembly for generating accurately controlled magnetic fields, the strength of which vary over a designated field of view (FOV) of the scanner.

The scanner 102 may include an RF coil 124 for generating RF pulses for exciting a gyromagnetic material of interest, typically bound in tissues of the patient 112. In some embodiments, the RF coil 124 may also serve as a receiving coil. Accordingly, the RF coil may be operationally coupled to transmit-receive circuitry 126 in passive and active modes for receiving emissions from the gyromagnetic tissue material and for applying RF excitation pulses, respectively. Alternatively, the system 100 may include various configurations of receiving coils, including, for example, structures specifically adapted for target anatomies, such as knee and/or chest coil assemblies.

In some embodiments, the system controller 104 controls operation of the associated MR coils for generating desired magnetic field and RF pulses. Accordingly, in some embodiments, the system controller 104 may include a pulse sequence generator 128, timing circuitry 130 and a processing subsystem 132 for generating and controlling imaging gradient waveforms and RF pulse sequences employed during patient imaging. In some embodiments, the system controller 104 may also include amplification circuitry 134 and interface circuitry 136 for controlling and interfacing between the pulse sequence generator 128 and the coils of the scanner 102. The amplification circuitry 134 may include one or more amplifiers that process the imaging gradient waveforms for supplying desired drive current to each of the gradient coils 118, 120 and 122 in response to control signals received from the processing subsystem 132. In some embodiments, the amplification circuitry 134 may also amplify and couple the generated RF pulses to the RF coil for transmission.

The processing subsystem 132 may include one or more digital and/or general purpose computer processors or other processing or custom-designed or configured components. In some embodiments, the processing subsystem 132 may, in addition to controlling the generation and capture of image data, process the response signals emitted by excited patient nuclei in response to the RF pulses.

The processing subsystem 132 may be configured to transmit image data to an image reconstruction unit 138 to allow reconstruction of desired images.

In some embodiments, the system controller 104 may include a storage repository 140 for storing acquired data, reconstructed images and/or information derived therefrom. In some embodiments the storage repository 140 may further include programming code for implementing image processing procedures as described in this disclosure.

In some embodiments, the system controller 104 may include interface components 142 for exchanging stored information such as scanning parameters and image data with the operator interface 106. In some embodiments, the operator interface 106 may allow an operator 144 to specify commands and scanning parameters.

In some embodiments, the operator interface 106 may also include output devices 148 such as a display 150 (e.g., one or more monitors) and/or one or more printers 152.

In some embodiments, image data derived from MRI images and/or MR scanning may be used in conjunction with PET image reconstruction and attenuation map generation. The PET data may be acquired sequentially and/or substantially simultaneously with the MR data acquisition. In some embodiments, a positron emitter or a radiotracer may be administered to the patient 112 that targets specific tissues or regions of the patient's body.

The system 100, in some embodiments, may include a detector ring assembly 154 disposed about the patient bore. The detector ring assembly 154 may be configured to detect radiation events corresponding to the target portion of the patient's body. The detector ring assembly 154 may include detector modules 156 that form detector rings included in the detector ring assembly 154. A set of acquisition circuits 158 in the system 100 may receive analog signals produced in the detector modules 156 and generate corresponding digital signals indicative of the location and energy associated with radiation events detected by the detector modules 156.

In some embodiments, the system 100 may include a data acquisition system (DAS) 160 that periodically samples the digital signals produced by the acquisition circuits 158. The DAS 160, in turn, includes event locator circuits 162 that assemble information corresponding to each valid radiation event into an event data packet. The event locator circuits 162 may communicate the event data packets to a coincidence detector 164 for determining coincidence events. The coincidence detector 164 may determine coincidence event pairs if time and location markers in two event data packets are within certain designated thresholds.

In some embodiments, the system 100 stores the determined coincidence event pairs in the storage repository 140. In some embodiments, the storage repository 140 includes a sorter 166 to sort the coincidence events in a 3D projection plane format, for example, using a look-up table. The processing subsystem 132 may process the stored data to determine time-of-flight (TOF) and/or non-TOF information. The image reconstruction unit 138 may be part of or separate from the processing subsystem 132.

Conventional PET imaging entails reconstruction of a PET activity map that defines a spatial distribution of a radiotracer in the patient body based on photons measured by detector modules 156. The emitted photons that travel through different regions of the patient body or other objects experience different attenuations. It is known to correct for these attenuation values to provide accurate PET quantitation in activity maps. One or more attenuation maps may be utilized for this purpose.

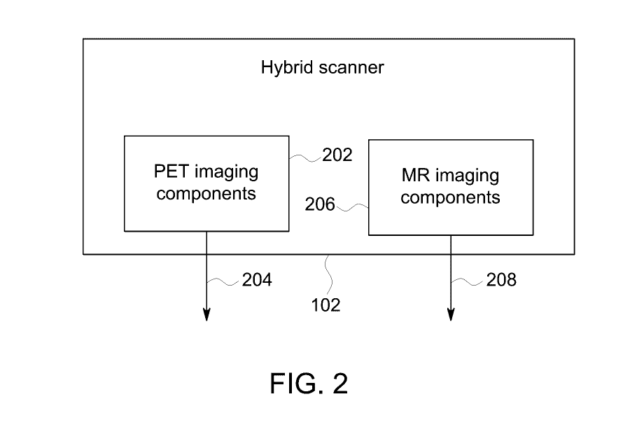

FIG. 2 is a block diagram that illustrates aspects of the scanner 102 in a different format. From FIG. 2, it will be observed that the scanner 102 includes PET imaging components 202 that produce emission data (indicated as a data stream at 204), and MR imaging components 206 that produce MR image data (indicated as a data stream at 208).

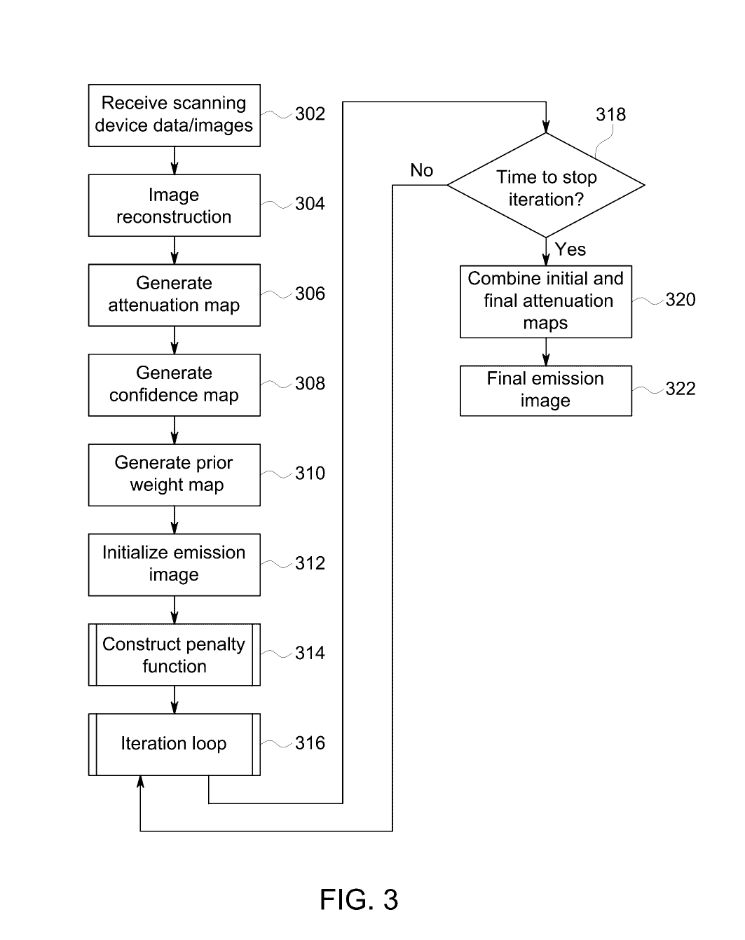

FIG. 3 is a flow chart that illustrates a process that may be performed in the imaging system of FIG. 1, according to some embodiments.

At 302 in FIG. 3, emission and/or MR image/scan data and/or images are received from the imagining components described above. In some embodiments, the MR scan data may be 3D gradient-echo (GRE) MR scan data with Dixon-type fat-water separation. In another embodiment, the MR scan data may be ZTE (zero-echo-time) MR scan data.

At 304 image reconstruction may occur. For example, such images may include fat, water, in-phase and out-of-phase images and/or ZTE (zero-echo-time) images.

At 306 an attenuation map is generated. The attenuation map may form an array of linear attenuation coefficients for 511 keV photons, and may be generated from the images obtained at 304. The attenuation map generation may be segmentation-based and/or atlas-based. Truncated regions, which may be due to smaller MR field-of-view (FOV) than PET FOV, may be completed using TOF non-attenuation corrected (NAC) PET images. Anatomy contexts may be used to reduce metal implant induced artifacts. Hardware attenuation (i.e., from table and rigid RF coils) may be used from pre-acquired templates. The attenuation map, as will be understood by those who are skilled in the art, may be considered a prior map, with the MR image(s) playing the role of a prior image in connection with subsequent image processing described herein.

At 308, a confidence map may be generated. The confidence map may represent the degree of confidence in each voxel of the attenuation image. Values in the confidence map may represent the accuracy in the prior map or the second source scan images such that large values are assigned in regions that are accurate in the prior map or the second source scan images, and small values are assigned in regions that are inaccurate in the prior map or the second source scan images. For example, the confidence map may be generated by converting the in-phase or ZTE MR image by a monotonic function such that a small value is assigned to a voxel in the confidence map if the corresponding MR image intensity is small. The monotonic function may be constant for some intervals. In some embodiments, fat and water MR images may be used to generate the confidence map. If the sum of fat and water MR signals in a voxel is sufficiently large, a large confidence value may be assigned to the voxel in the confidence map. In an alternative embodiment, if air is segmented in MR images, a large confidence value may be assigned to the voxels corresponding to the air segments. Similarly, if some anatomical organs such as lungs are segmented or identified in MR images, a large confidence value may be assigned to those regions. The confidence map may be binary-valued or continuous-valued. In some embodiments, the binary-valued confidence map may be obtained by thresholding.

At 310, a prior weight map may be generated. This may, for example, be done by converting the confidence map generated at 308 by a monotonic function. In such a case, a small value may be assigned to a voxel in the prior weight map if the corresponding value in the confidence map is small, and a large value may be assigned to a voxel in the prior weight map if the corresponding value in the confidence map is large. In an alternative embodiment, the prior weight map may be uniform. In some embodiments, a body contour may be incorporated into the prior weight map. Large values may be assigned to the voxels in the prior weight map outside the body contour. In the prior weight map, large values may also be assigned to the voxels close to the body contour. The body contour may be obtained from TOF non-attenuation corrected PET images, MR images, and/or PET images that are reconstructed using the attenuation map generated at 306. In an alternative embodiment, the prior weights may be spatially modulated according to PET sensitivities. Generally, PET sensitivities are axially decreasing from the central slice to end slices because of a variation in the number of lines of response passing through each slice, and PET sensitivities are trans-axially increasing from the center of the trans-axial FOV towards the body boundary. In some embodiments, large values may be assigned to some organs such as bladders and hearts and/or high activity regions in the prior weight map. Such organs and/or high activity regions may be obtained using TOF non-attenuation corrected PET images, MR images, and/or PET images that are reconstructed using the attenuation map generated at 306. The prior weight map may be binary-valued or continuous-valued. In some embodiments, the binary-valued prior weight map may be obtained by thresholding.

At 312, an emission image (activity image) may be initialized. For example, the initial emission image may be a uniform image.

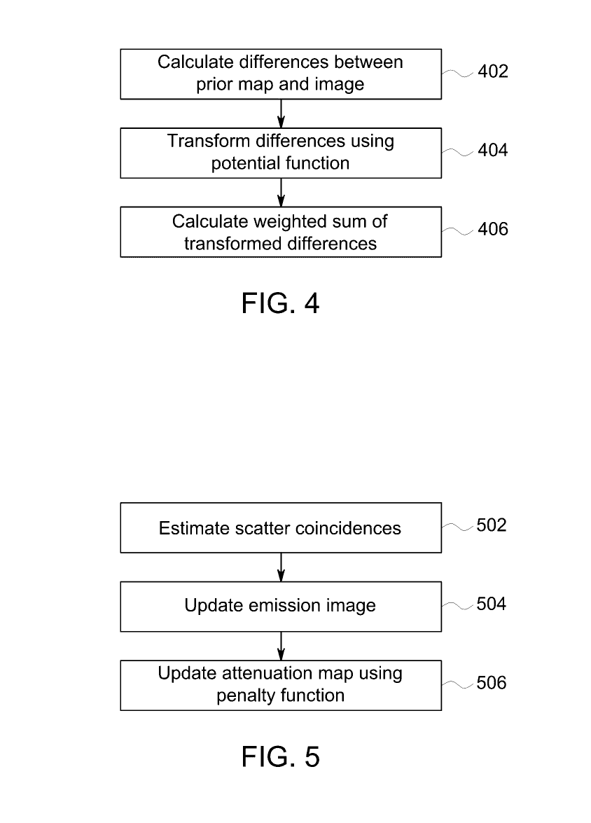

At 314, a penalty function may be constructed. FIG. 4 is a flow chart that illustrates characteristics of an example penalty function that may be constructed at 314.

Turning then to FIG. 4, at 402, differences between a current version of the attenuation map and the prior map generated at 306 are calculated. At 404, the differences between the current version of the attenuation map and the prior map are transformed using a potential function. At 406, a weighted sum of the transformed differences is calculated. Details of example potential and penalty functions will be described below in connection with a discussion of iterative updating of the attenuation map and the activity image.

Referring again to FIG. 3, at 316, an iteration loop is performed for updating the attenuation map and the activity image. The processing at 316 involves joint reconstruction of activity and attenuation using emission data, while also synergistically incorporating prior information from the MR scan data. The attenuation map may be initialized based on the prior map generated at 306. FIG. 5 is a flow chart that illustrates details of the iteration loop 316.

Referring now to FIG. 5, at 502, scattered coincidences may be estimated by a known technique such as model-based scatter estimation. At 504, the emission image is updated based on the current version of the attenuation map and the emission projection data. At 506, the attenuation map is updated based on the current version of the emission image and the emission projection data using the penalty function.

Referring again to FIG. 3, as indicated by decision block 318, at some point it is determined that the performance of the iteration loop 316 is to cease. In some embodiments, this may occur after a fixed, pre-determined number of iterations. In some embodiments, this may occur when the differences between the most recent emission image and/or attenuation map, and the corresponding results of the previous iteration differ by less than a threshold amount.

An example embodiment of the iteration loop 316 will now be described in which a fixed, pre-determined number of iterations is employed. At a high level, the loop may be summarized as follows:

For nᵢₜₑᵣ=1:Nᵢₜₑᵣ

(Step 1) Update the activity image by TOF OSEM (ordered subset expectation maximization—a known technique).
(Step 2) Update the attenuation map by OSTR (ordered subset transmission). End

Details of the OSTR algorithm as performed according to some embodiments will be described below. In this example embodiment, Nᵢₜₑᵣ=5 may be used. In Step (1), 2 iterations may be used with 28 subsets for TOF OSEM. In Step (2), 10 iterations may be used with 28 subsets for the OSTR algorithm. In some embodiments, alternative algorithms may be used in Step (1) such as OSEM or penalized likelihood or regularized reconstruction algorithms, and/or alternative algorithms may be used in Step (2) such as gradient methods or Newton's methods. In other embodiments, time-of-flight emission projection data may be used in Step (1) and non-time-of-flight emission projection data may be used in Step (2). Non-time-of-flight emission projection data may be obtained by summing time-of-flight emission projection data across time-of-flight bins. In another embodiments, in Step (1) and/or Step (2), time-of-flight emission projection data may be used until the iterative updates are performed a predetermined number of times, and non-time-of-flight emission projection data may be used after the iterative updates are performed the predetermined number of times.

In the OSTR algorithm, according to some embodiments, the following regularization function may be applied to the attenuation map μ.

R(μ)=R_{coughness}(μ)+R_{MR}(μ)

The roughness penalty R_{roughness}(μ) penalizes the squared difference between neighboring voxel pairs according to the following formula.

R_{roughness}(μ)=β_{roughness}ρ_{j,k:neighbors}wⱼₖ(μⱼ−μₖ)²

For the preceding formula, wⱼₖ∈{1, (sqrt(2))⁻¹, (sqrt(3))⁻¹} are weights determined by the distance between voxels j and k; the penalty strength β_{roughness} may be chosen as 2×10⁴. In some other embodiments, non-quadratic functions may be used for the roughness penalty and/or the penalty weights may be spatially modulated according to sensitivities. The MR-based prior R_{MR} penalizes the deviation from the MR-based attenuation map μ^{MR}—that is, the prior map; the following formula is applicable.

R_{MR}(μ)=β_{MR}ΣⱼγⱼΩ(μⱼ−μⱼ^{MR})

For the preceding formula, β_{MR} may be an MR-based prior strength parameter, which in some embodiments may be chosen as β_{MR}=10⁵; Ψ may be a potential function, which in some embodiments may be a quadratic function Ψ(t)=t²; γⱼ may be modulation factors, which represents a prior weight map; in some embodiments γⱼ=10⁻² may be used when voxel j belongs to the low MR signal region; and otherwise γⱼ=1 may be used; in this case, γⱼ is binary-valued. In an alternative embodiment, γⱼ may be continuous-valued such that γⱼ is a function of the MR signal intensity in voxel j where the function is monotonically increasing. In some embodiments, non-quadratic functions may be chosen for the potential function Ψ. The μ^{MR} may represent the prior map generated at 306; γⱼ or β_{MRγj} may represent the prior weight map generated at 310; and R_{MR}(μ) may represent the penalty function constructed at 314.

In some embodiments, the MR-based prior weight may be modulated such that it increases towards the edge of the trans-axial FOV or the outer boundaries of the body.

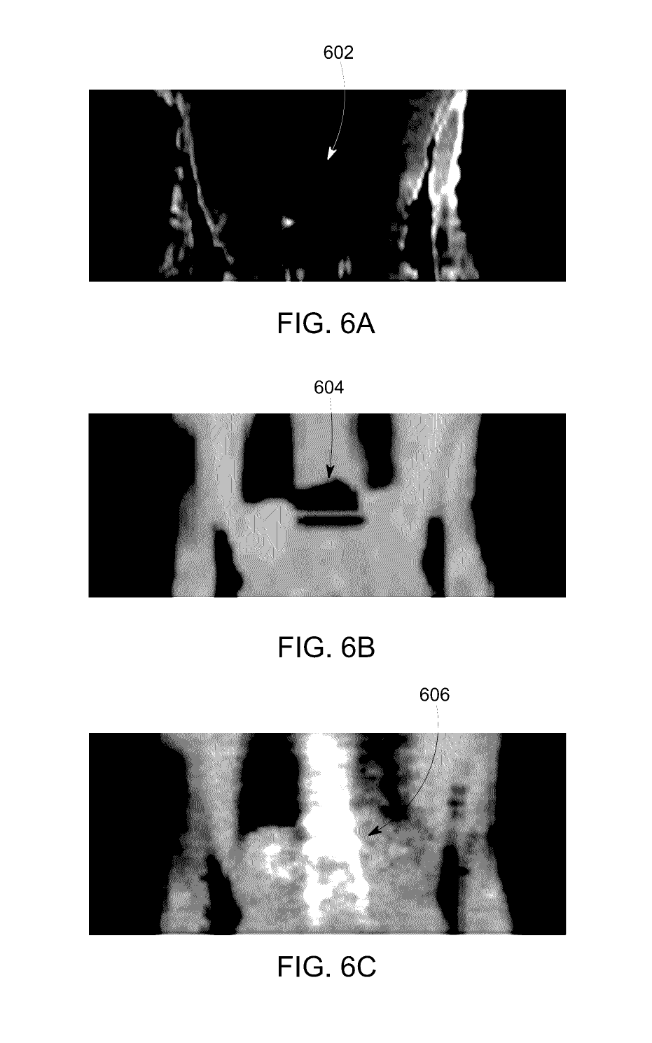

Representative results of the reconstruction approach described above are illustrated in FIGS. 6A-6C, 7A-7C, 8A-8C, 9A-9C, 10A-10C and 11A-11C.

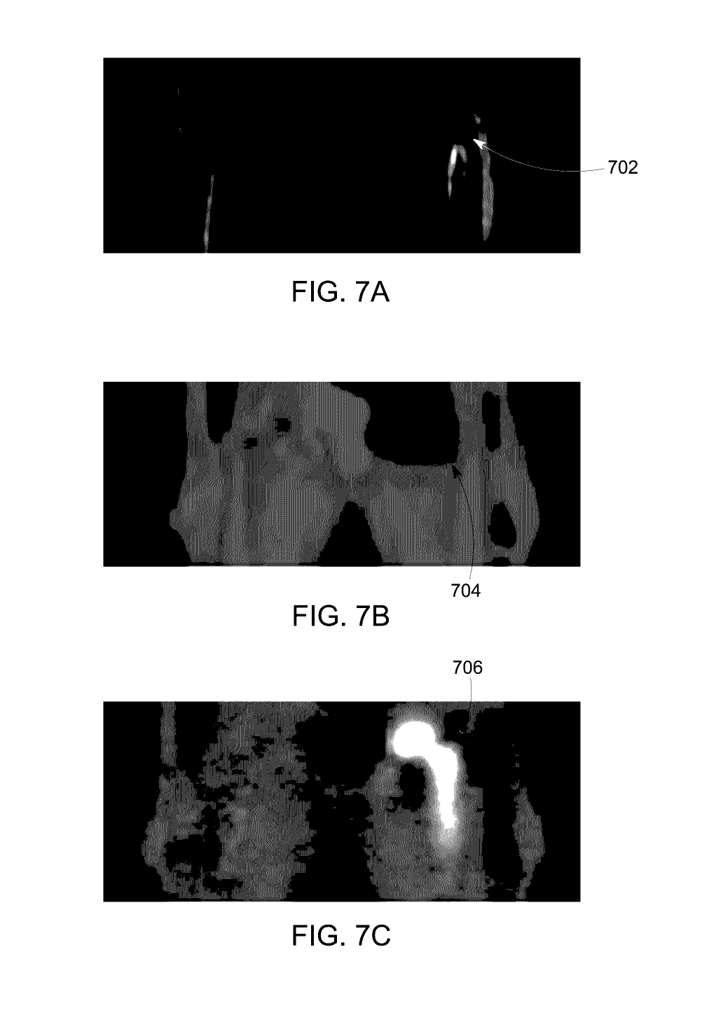

For example, FIG. 6A shows a dark region 602 (or a low MR signal region) in an MR (in-phase) image and FIG. 6B shows a corresponding dark region 604 in an MR-based attenuation map (or a prior map), in both cases resulting from spinal implants. Similar dark regions, due to hip implants, are shown at 702 in FIG. 7A (MR image) and at 704 in FIG. 7B (MR-based attenuation map or prior map). A joint reconstruction approach according to embodiments of this disclosure allows the implants to be recovered in the attenuation maps, as indicated at 606 in FIG. 6C and at 706 in FIG. 7C.

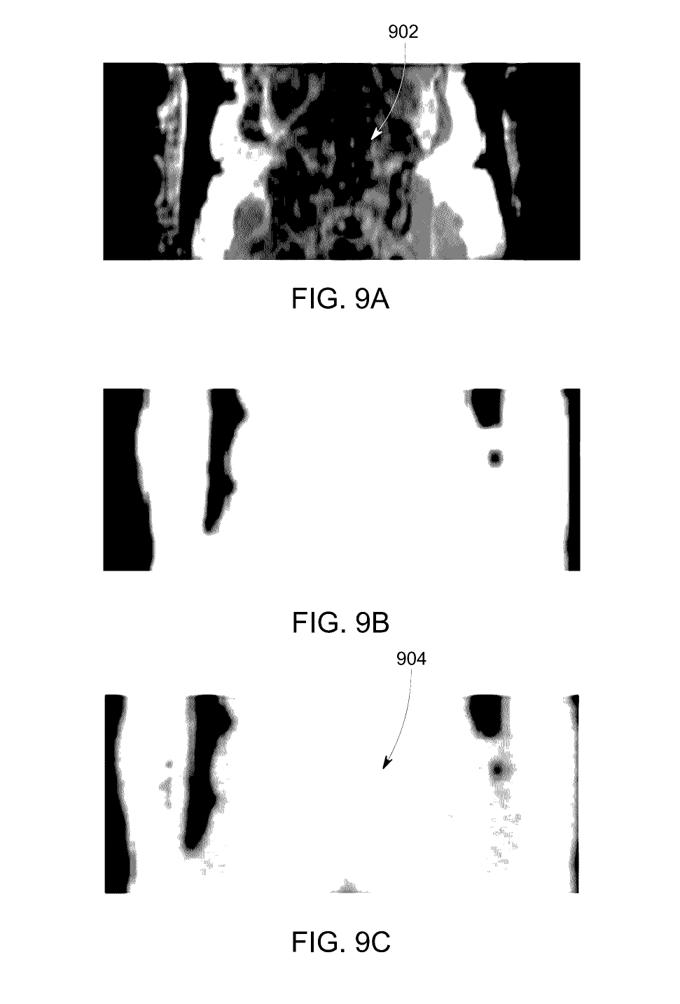

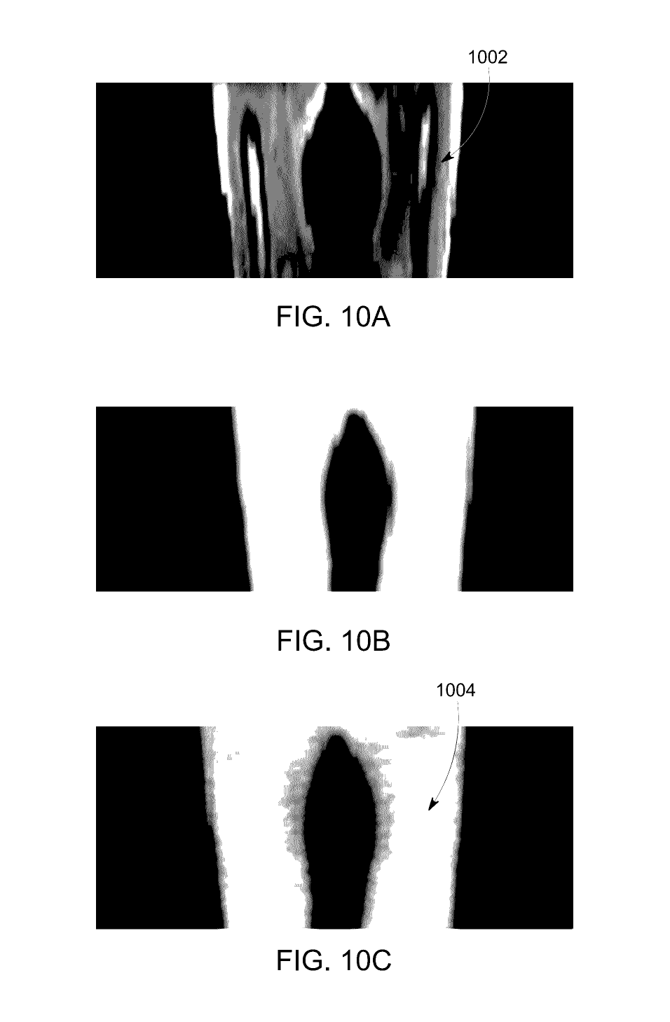

Bones (seen at 902 in FIG. 9A and at 1002 in FIG. 10A) may be missing from the corresponding MR-based attenuation maps (FIGS. 9B and 10B, respectively); but nevertheless may be recovered via the joint reconstruction approach according to some embodiments (reference numeral 904—spinal bones, FIG. 9C; reference numeral 1004—leg bones, FIG. 10C).

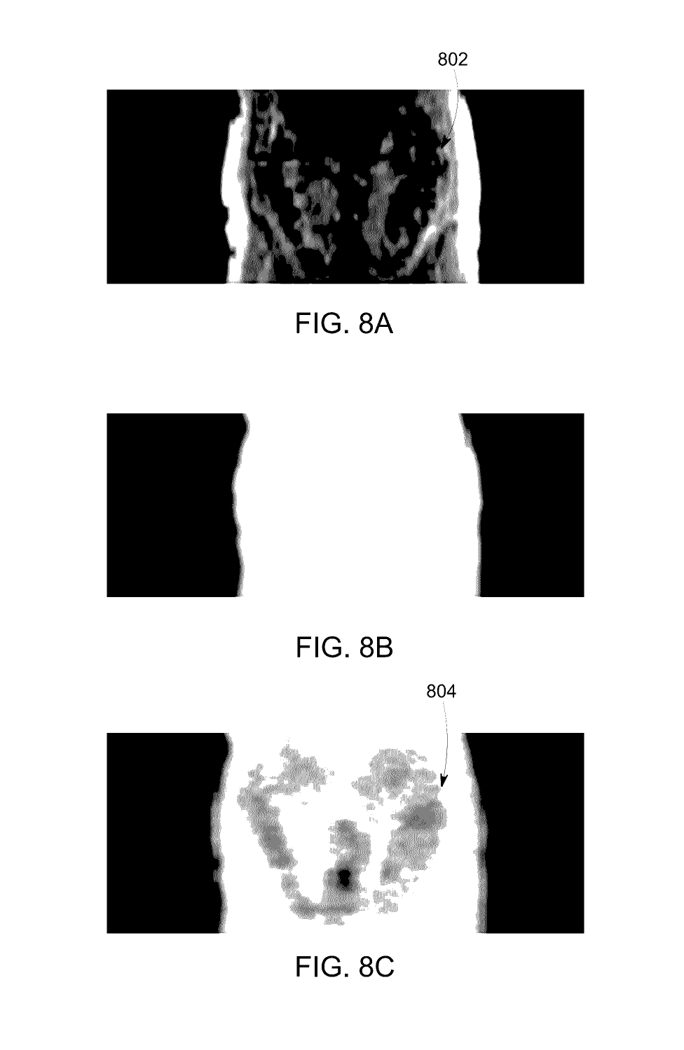

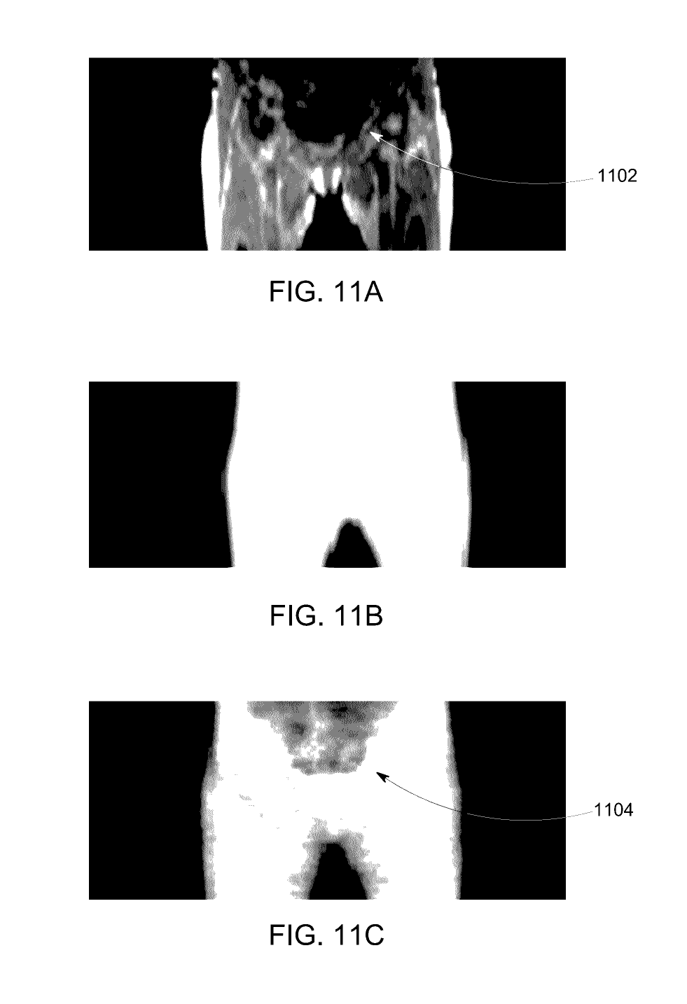

By the same token, internal air cavities (seen at 802 in FIG. 8A and at 1102 in FIG. 11A) may again be missing from the corresponding MR-based attenuation maps (FIGS. 8B and 11B, respectively); but may be recovered via the joint reconstruction approach according to some embodiments, as indicated at 804 in FIG. 8C and at 1104 in FIG. 11C.

Referring again to FIG. 5, in some embodiments, step 502 (estimation of scatter coincidences) may be performed only once (i.e., prior to the iteration loop 316, FIG. 3), and hence may be omitted from the process illustrated in FIG. 5.

Referring again to FIG. 3, and particularly decision block 318, the process of FIG. 3 may continue looping back from decision block 318 to the iteration loop 316 until it is determined that it is time to stop performing the iteration loop. That determination may be made, for example, based on a predetermined number of iterations having been performed, or based on the change in image/attenuation map resulting from the latest iteration being below a predetermined threshold. Upon a positive determination being made at decision block 318 (i.e., upon determining that it is time to stop performing the iteration loop 316), then block 320 may follow decision block 318. At block 320, the final attenuation map resulting from the processing at 316 may be combined with the initial attenuation map generated at 306. For example, a weighted averaging of the two attenuation maps may be performed. The weights for the weighted averaging may be determined using the confidence map or the prior weight map. In another embodiment, the final attenuation map may be the attenuation map updated in the last iteration of the loop 316.

Block 322 may follow block 320. At block 322, a final emission/activity image may be reconstructed using the attenuation map formed at 320. In another embodiment, the final emission image may be the emission image updated in the last iteration of the loop 316.

In some embodiments, the binary-valued prior weight map generated at 310 may be filtered so that the prior weight map is smooth. In some other embodiments, a smooth prior weight map is generated at 310 by having smooth transitions from low confidence regions to high confidence regions. In another embodiment, the continuous-valued prior weight map generated at 310 may be filtered.

In some embodiments, steps 308 and/or 320 may be omitted from the process illustrated in FIG. 3. Where step 308 is omitted, the prior weight map may be generated from the MR image.

In some embodiments, rather than using monotonic functions at steps 308 and/or 310, non-monotonic functions may be used. For example, the latter function or functions may be mainly monotonic, but not monotonic in certain intervals.

In example embodiments described above, PET was employed as a source of emission projection data and MR was employed as a source of prior information (i.e., a second source of scan data). However, in other embodiments, for example, SPECT (single-photon emission computed tomography) or optical luminescence are possible alternative sources of emission projection data. Moreover, in some embodiment a CT (computerized tomography) scan is a possible alternative second source of scan data. In another embodiment, atlas or template images may be used as a second source scan data. In this case, reconstructing second source images may amount to registering the atlas or template images and/or performing necessary image processing operations.

In embodiments described above, a joint reconstruction based on emission data also uses MR-based priors. The MR-based prior weights are spatially modulated to rely more on joint reconstruction in low MR signal regions, and more on the MR-based priors in soft-tissue regions, which MR is good at imaging. Results have indicated that image processing according to embodiments of this disclosure can recover the attenuation of implants, bones and internal air cavities. The MR-based priors are simple and may be effective for multiple patients in a robust way.

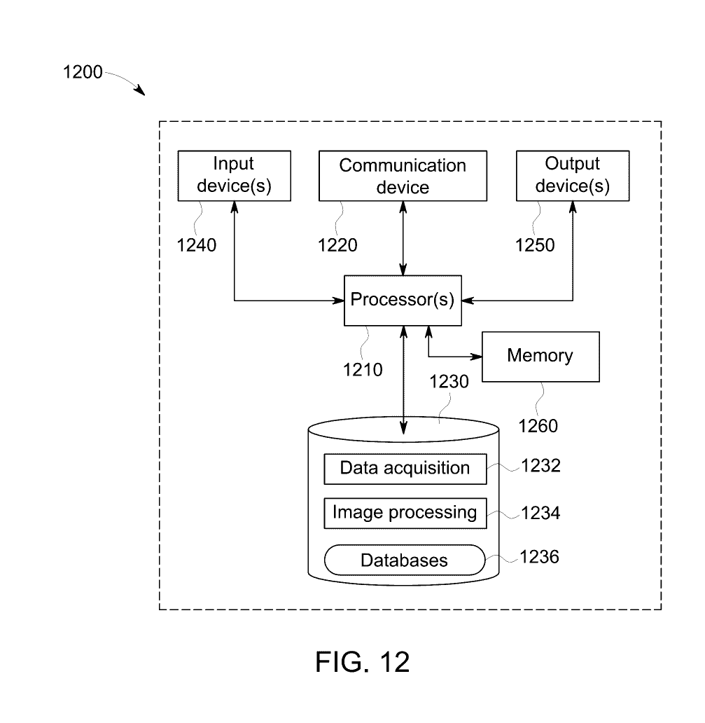

FIG. 12 shows a computer 1200 that may constitute at least some portions of the system controller 104 (FIG. 1) and/or other components of the system 100. Continuing to refer to FIG. 12, computer 1200 includes one or more processors 1210 operatively coupled to communication device 1220, data storage device 1230, one or more input devices 1240, one or more output devices 1250 and memory 1260. Communication device 1220 may facilitate communication with external devices, such as other components of the system 100 (FIG. 1) and/or remote computers to which diagnostic images are to be downloaded. Continuing to refer to FIG. 12, input device(s) 1240 may include, for example, a keyboard, a keypad, a mouse or other pointing device, a microphone, knob or a switch, an infra-red (IR) port, a docking station, and/or a touch screen. Input device(s) 1240 may be used, for example, to enter information into the computer 1200. Output device(s) 1250 may include, for example, a display (e.g., a display screen) a speaker, and/or a printer.

Data storage device 1230 may include any appropriate persistent storage device, including combinations of magnetic storage devices (e.g., magnetic tape, hard disk drives and flash memory), optical storage devices, Read Only Memory (ROM) devices, etc., while memory 1260 may include Random Access Memory (RAM).

Data storage device 1230 may store software programs that include program code executed by processor(s) 1210 to cause computer 1200 to perform any one or more of the processes described herein. Embodiments are not limited to execution of these processes by a single apparatus. For example, the data storage device 1230 may store an image data acquisition software program 1232.

Data storage device 1230 may also store an image data processing software program 1234, which may, for example, provide functionality that corresponds to the processes described above in connection with FIGS. 3-5. Further, data storage device 1230 may store one or more databases 1236. Data storage device 1230 may store other data and other program code for providing additional functionality and/or which are necessary for operation of computer 1200, such as device drivers, operating system files, etc.

A technical effect is to provide improved processing of diagnostic emission projection images.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method, comprising:
receiving emission projection data and second source scan data corresponding to a subject, said second source scan data from a mode of imaging different from emission projection imaging;
reconstructing second source images based on the second source scan data;
generating a prior map based on the second source images;
generating a prior weight map, comprising:
generating a confidence map based on the second source images; and
generating a prior weight map that is spatially varying based on the confidence map;
constructing a penalty function that
calculates voxel-wise differences between the prior map and a given image;
transforms each voxel-wise difference by using a potential function; and
calculates a weighted sum of the transformed voxel-wise differences where weights for the weighted sum are based on the prior weight map;
reconstructing an emission image and an attenuation map, comprising:
iteratively updating the emission image based on the attenuation map and the emission projection data;
iteratively updating the attenuation map based on the emission image and the emission projection data by using the penalty function;
obtaining a final attenuation map; and
generating a final emission image.

2. The method of claim 1, wherein said second source scan data is magnetic resonance scan data.

3. The method of claim 1, wherein the prior weight map is binary-valued.

4. The method of claim 1, wherein the prior weight map is continuous-valued.

5. The method of claim 1, further comprising:
estimating scattered coincidences in the emission projection data.

6. The method of claim 1, wherein said steps of iteratively updating the emission image and iteratively updating the attenuation map are performed a predetermined number of times.

7. The method of claim 1, further comprising:
detecting a degree of change in at least one of said updated emission image and said updated attenuation map due to a most recent iteration of one or both of said updating steps; and
ceasing said iteratively updating steps based on a comparison of said detected degree or degrees of change with at least one threshold value.

8. The method of claim 1, wherein the steps of generating the confidence map and/or the prior weight map include at least one of:
applying thresholding to the second source images;
transforming the second source images by using a monotonic function;
segmenting organs or uniform regions in the second source images;
using anatomical knowledge; and
spatially modulating the prior weight map.

9. The method of claim 8, wherein the step of spatially modulating the prior weight map is based on at least one of:
emission sensitivities;
emission images that are reconstructed without attenuation correction or based on the prior map; and
body contours obtained from the second source images and/or the emission images that are reconstructed without attenuation correction or based on the prior map.

10. The method of claim 1, further comprising:
initializing an attenuation map based on the prior map.

11. The method of claim 1, wherein, for the step of iteratively updating the emission image based on the attenuation map and the emission projection data, the emission projection data are time-of-flight emission projection data; and
wherein, for the step of iteratively updating the attenuation map based on the emission image and the emission projection data, the emission projection data are non-time-of-flight emission projection data.

12. The method of claim 1, wherein, for the step of iteratively updating the emission image based on the attenuation map and the emission projection data, the emission projection data are time-of-flight emission projection data until said step of iteratively updating the emission image is performed a predetermined number of times, and the emission projection data are non-time-of-flight emission projection data after said step of iteratively updating the emission image is performed the predetermined number of times.

13. An imaging apparatus, comprising:
a first imaging device for producing emission projection data corresponding to a subject;
a second imaging device for providing second source scan data corresponding to the subject, said second imaging device different from said first imaging device; and
a computer coupled to the first and second imaging devices; the computer comprising a processor and a memory in communication with the processor, the memory storing program instructions, the processor operative with the program instructions to perform functions as follows:
receiving the emission projection data and the second source scan data;
reconstructing second source images based on the second source scan data;
generating a prior map based on the second source images;
generating a prior weight map, comprising at least one of:
applying thresholding to the second source images;
transforming the second source images by using a monotonic function;
segmenting organs or uniform regions in the second source images using anatomical knowledge; and
spatially modulating the prior weight map;
constructing a penalty function that
calculates voxel-wise differences between the prior map and a given image;
transforms each voxel-wise difference by using a potential function; and
calculates a weighted sum of the transformed voxel-wise differences where weights for the weighted sum are based on the prior weight map;
reconstructing an emission image and an attenuation map, comprising:
iteratively updating the emission image based on the attenuation map and the emission projection data;
iteratively updating the attenuation map based on the emission image and the emission projection data by using the penalty function;
obtaining a final attenuation map; and
generating a final emission image.

14. The apparatus of claim 13, wherein the first imaging device is a PET (positron emission tomography) scanner.

15. The apparatus of claim 13, wherein the first imaging device is a SPECT (single photon emission computed tomography) scanner.

16. The apparatus of claim 13, wherein the first imaging device is an optical luminescence scanning device.

17. The apparatus of claim 13, wherein the second imaging device is a magnetic resonance scanner.

18. The apparatus of claim 13, wherein the prior weight map is binary-valued.

19. The apparatus of claim 13, wherein the prior weight map is continuous-valued.

20. The apparatus of claim 13, wherein said functions of iteratively updating the emission image and iteratively updating the attenuation map are performed a predetermined number of times.

21. The apparatus of claim 13, wherein:
the processor is further operative with the program instructions to detect a degree of change in at least one of said updated emission image and said updated attenuation map due to a most recent iteration of one or both of said updating functions; and
the processor is further operative with the program instructions to cease said iteratively updating functions based on a comparison of said detected degree or degrees of change with at least one threshold value.

22. The apparatus of claim 13, wherein the step of spatially modulating the prior weight map is based on at least one of:
emission sensitivities;
emission images that are reconstructed without attenuation correction or based on the prior map; and
body contours obtained from the second source images and/or the emission images that are reconstructed without attenuation correction or based on the prior map.

23. A method comprising:
obtaining emission projection data;
obtaining second source images based on second source scan data, said second source scan data from a mode of imaging different from a mode employed to obtain the emission projection data;
generating a first attenuation map from said second source scan data;
generating a confidence map for said attenuation map;
generating a prior weight map based on at least one of said emission projection data, said confidence map and said second source images;
constructing a penalty function that
calculates voxel-wise differences between the attenuation map and a given image;
transforms each voxel-wise difference by using a potential function; and
calculates a weighted sum of the transformed voxel-wise differences where weights for the weighted sum are based on the prior weight map;
reconstructing updated versions of an emission image and the first attenuation map, comprising:
iteratively updating the emission image based on a current version of the attenuation map and the emission projection data;
iteratively updating the current version of the attenuation map based on the emission image and the emission projection data by using the penalty function;
determining a point at which to cease said updating steps; and
ceasing said updating steps based on a result of said determining step;
obtaining a final attenuation map based on a final iteration of said step of iteratively updating the attenuation map;
forming an averaged attenuation map as a weighted average of the final attenuation map and the first attenuation map; and
generating a final emission image.

24. The method of claim 23, wherein the step of forming an averaged attenuation map uses weights determined based on said confidence map.

25. The method of claim 23, wherein the prior weight map is generated from the confidence map using a monotonic function.

26. The method of claim 23, wherein the second source images are magnetic resonance images.
